(19) United States
(12) Patent Application Publication
(10) Pub. No.: US 2004/0167395 A1
(43) Pub. Date: Aug. 26, 2004

(54) Dynamic medical imaging

(75) Inventors: Christian Peter Behrenbruch, Oxford (GB); Jerome Marie Joseph Declerck, Oxford (GB); John Michael Brady, Oxford (GB)

Correspondence Address: KALOW & SPRINGUT LLP, 488 MADISON AVENUE, 19TH FLOOR, NEW YORK, NY, 10022, US

(73) Assignee: MIRADA SOLUTIONS LIMITED, British body corporate, Oxford (GB)

(21) Appl. No.: 10/756,953

(22) Filed: Jan. 13, 2004

(30) Foreign Application Priority Data
Jan. 15, 2003 (GB) 0300921.4

(51) Int. Cl. A61B 5/05; A61B 6/00

(52) U.S. Cl. 600/420; 600/431

(57) ABSTRACT

A method of dynamic medical imaging in which the quality of image registration or motion correction between the frames is assessed by examining the temporal behaviour of a region of interest through the sequence, and in particular how closely the behaviour follows expected behaviour. For a temporal sequence a temporal model of the expected behaviour of the region of interest is available and the quality of fit between the model and the data points from the dynamic imaging sequence can be calculated. In image regions of high patient motion the fit will be poor, whereas if there is no patient motion the fit will be better. The quality of fit can be displayed on the image as an indication of the validity of motion correction, and in areas of poor fit the motion correction can be re-executed using different parameters to try and improve the fit between the behaviour model and the actual data points. The invention is applicable to any contrast-enhanced medical imaging technique.

### BACKGROUND

The present invention relates to dynamic medical imaging, that is to say the acquisition of a plurality of time separated images of a subject in order to reveal some aspect of the medical condition of the subject.

Medical imaging is a technology that has evolved substantially over the past few decades with an increasing diversity of modalities—Magnetic Resonance Imaging (MRI), Computed Tomography (CT), Nuclear Medicine (NM) and Positron Emission Tomography (PET)—to name but a few. Although the traditional notion of medical imaging involves the acquisition of a “static” image that captures the anatomy of an organ/region of the body, increasingly the use of more sophisticated imaging techniques allows dynamic studies to be made which provide a temporal sequence of images which can characterise physiological or pathophysiological information.

Both static and dynamic medical imaging may involve the use of an imaging technique to increase selectively the contrast of a certain region in the image (e.g. on the basis of that region's structure or physiological state). For example, one may inject into a patient a compound which has a biophysical, molecular, genetic or cellular affinity for a particular organ, disease, state or physiological process. Such contrast agents are selected to have a property that provides enhanced information to a given imaging modality by altering imaging conditions (normally by altering the contrast) to reflect the behaviour of the compound in the body. This may be achieved via increased X-ray attenuation a localised site (e.g. for CT/X-ray), altered paramagnetic properties (e.g. for MRI) or the use of a radioisotope for nuclear medicine/PET. Such contrast agents are well-known for many imaging modalities. In some cases a technique may be used to create a contrast enhancement which does not rely on an injected contrast agent, for instance the use of “black blood” or “white blood” in magnetic resonance (MR) imaging (where specific pulse sequences are used to change the magnetic saturation of the blood and thus its appearance in the image), or tagged MR sequences which alter the magnetic behaviour of particular tissue or fluid. An injected contrast agent, or a tissue or fluid with altered behaviour, may all be regarded as “imaging agents”.

Analysis of the behaviour of an imaging agent through imaging typically involves an acquisition process that takes many “snapshots” of the organ/region/body over time in order to capture the time-varying behaviour, e.g. distribution, of the agent and hence capture a specific biological state (disease, condition, physiological phenomenon, etc.). As the speed and digital nature of medical imaging evolves, this acquisition data can have tremendous temporal resolution and results in large quantities of data.

Additionally, there is a growing interest in not only imaging a particular physiological phenomenon to characterise a disease or condition, but also the in vivo assessment of the change in disease in response to therapy. This therapy may be surgical, pharmacological/genetic or radiotherapeutic in nature. Whether for diagnosis or treatment assessment purposes, dynamic imaging is a powerful clinical tool and has not only altered the landscape of medical imaging but has also made substantial inroads into drug discovery and animal applications.

Irrespective of the application, dynamic imaging with an imaging agent requires a temporal (multiple time epoch) acquisition during which the behaviour of the imaging agent is characterised. Sometimes this characterisation involves known physiological, cellular or genetic models that may have arbitrary interaction complexity. Sometimes this characterisation is performed using mathematical or statistical models that abstract true physiological meaning but which may enable certain parameters to be calculated which indicate the behaviour of the imaging agent and hence provide diagnostic information.

A patient may need to spend considerable time in an imaging device in order to acquire an image sequence. During this time, a patient may move—either due to natural bodily motion (respiration, “physiological tremor”, etc) or as part of a symptom of disease (e.g. lack of motor control by an Alzheimer's patient). This motion necessarily corrupts the temporal accuracy of the imaging process, making it difficult to compare and, more critically, analyse individual regions of the imaging process.

Algorithms and clinical methods to address motion have long been a focus of research in medical imaging. Various techniques for motion correction, often involving registering images together to match corresponding locations in the image to each other, have been proposed, see for example WO 00/57361 and D. Rueckert, C. Hayes, C. Studholme, P. Summers, M. Leach, D. Hawkes, “Non-Rigid Registration of Breast MR Images Using Mutual Information”, in Proc. MCCAI (Medical Image Computing and Computed Assisted Intervention), 1998, pp. 1144-1152, ISBN 3-540-65136-5, Springer Verlag. However such methods have failed to address the following issues:

1) The validity of the motion correction.

2) Feedback to the user about the quality of the motion correction in a region of interest.

3) Feedback to the motion correction process itself regarding correction quality and analytical usefulness of the motion correction data.

In virtually all dynamic imaging studies that involve an imaging agent, the behaviour of the agent can be mathematically characterised at some level or another. In the most simplistic fashion the behaviour of an imaging agent such as an injected compound may be analysed using a model of “uptake” and possibly subsequent “wash out”, which may be linear or non-linear in nature. In more sophisticated instances, biophysical and pharmacokinetic understanding enables an imaging agent to be analysed using complex models that describe many different phases of behaviour and interaction. This is illustrated in FIG. 1 of the accompanying drawings, which show a dynamic MR imaging sequence of the breast as a contrast agent is injected. The left breast (on the right in the displayed coronal slice) is the region of interest, where the suspicious area needs clinical investigation using contraced-enhanced MRI. FIG. 1(a) illustrates the baseline image, that is to say with no imaging agent. FIG. 1(b) schematically represents the injection of contrast agent and the enhancement of the breast starts in FIG. 1(c), continues in FIG. 1(d) and finishes later in FIG. 1(e) with high enhancement of the breast. FIG. 1(f) represents the amount of image enhancement of the circled region in the image of FIGS. 1(a), (c), (d) and (e) plotted as a function of time. This can be fitted to the expected mathematical model of the uptake and washout rates of this contrast agent and from the fitted curve a value for the uptake rate and washout rate can be calculated. These values can be used to characterise the tissue in the circled region as cancerous or normal.

However, FIG. 1 illustrates schematically the ideal case where there is no patient movement. If the patient moves, this adds noise or corrupts the imaging information that is used for this dynamic analysis. Without motion correction, it can be understood that this will change the values for apparent image enhancement at any particular part of the breast (because the values will not be referring to the same part of the breast before and after movement). Thus if patient motion occurs it is no longer possible to analyse accurately the temporal characteristics of an imaging process.

FIG. 2 illustrates two sequences of images, a first in which motion is not corrected and a second in which motion is corrected. In the first sequence FIG. 2(a) is a baseline (i.e. before injection of contrast agent) magnetic resonance image of another breast clinical case. FIG. 2(b) shows the image after a gadolinium contrast agent has been injected. An easy way to judge the amount of enhancement, and thus isolate the tumourous region, is to subtract the two images of FIGS. 2(a) and 2(b). This results in the subtraction image of FIG. 2(c). Unfortunately because the patient moved between the pre-contrast and post-contrast images of FIGS. 2(a) and 2(b), the subtraction image of FIG. 2(c) contains many artefacts that suggest enhancement, but are in fact erroneous. One of the known motion correction techniques of registering the pre- and post-contrast images to each other can be used to find the motion field in the image. A suitable technique is disclosed in WO 00/57361 (herein incorporated by reference). The resulting motion field is shown in FIG. 2(d). It can be seen that there is significant motion in the right-hand part of the breast, but no motion in the left-hand part. The post-contrast image can be motion corrected to remove the movement resulting in the corrected image of FIG. 2(e), and then this is subtracted from the pre-contrast image of FIG. 2(a). The corrected subtraction image is shown in FIG. 2(f) and it can be seen that many motion artefacts have been removed (for example the highly visible artefacts around the edge of the breast in FIG. 2(c)), and the region corresponding to contrast enhancement (for instance tumours) within the breast is more clearly visible.

Although such motion correction techniques are useful, they are often regarded with suspicion by clinicians. This is especially true of non-rigid, or deformable, corrections where clinicians worry that the changes introduced by the motion correction may disguise clinically significant features. This has lead to a slow take-up in practice of the advantages given by registration techniques.

### SUMMARY OF THE INVENTION

The present invention provides a method of dynamic medical imaging in which the expected temporal characteristics of the imaging process, e.g. the model of behaviour of the imaging agent, is used to optimise a motion correction process, such as one of the known methods of registration, and to provide feedback to the user as to the quality of motion correction.

Thus the knowledge about the temporal behaviour of the imaged region can be used not only in the conventional diagnosis, but also to validate the motion correction. Further, if the motion correction is found by this method to be poor, it provides a way of identifying regions within the subject where the motion correction is poor, allowing them to be processed further, for instance using different parameters in the motion correction method. The invention also provides quality-control information to the user of the dynamic/temporal medical imaging study.

In more detail the present invention provides a method of dynamic medical imaging comprising the steps of:—

obtaining a plurality of time separated images of the subject;

registering the plurality of time separated images together to match corresponding locations in the images to each other;

measuring from the registered images the temporal behaviour of an imaged region at a location in the subject; and

comparing the measured temporal behaviour with a model of the expected temporal behaviour of the imaged region to determine the level of agreement therebetween as a measure of the quality of the registration of the time separated images.

Thus the expected behaviour of the imaged region is used to measure the quality of the registration.

The images may be acquired in a process involving the use of an imaging agent. The imaging agent may be the subject's own tissue or fluid or a contrast agent which is administered to the subject as mentioned above.

The level of agreement between the measured and expected temporal behaviour may be displayed to the user, for instance overlying an image of the subject, for example as a colour wash with different colours or intensities indicating the level of agreement.

The method may further include the step of re-executing the image registration in regions of the image where the level of agreement is poor. This may be achieved by using different parameters, such as using a different resolution, different scale or different sized search windows in the registration process.

The model may be a temporal model of the take-up and wash-out of an imaging agent and the subject of the imaging process may be a living human, animal or plant.

The technique is applicable to any dynamic imaging technique such as magnetic resonance imaging, computed tomography, positron emission tomography, nuclear medicine, ultrasound, x-ray and optical imaging.

The invention may be embodied as a computer program comprising program code means for executing the registration, measurement and comparison steps on a programmed computer, and the invention extends to a computer readable storage medium carrying such a program and to a computer system programmed to execute the method.

### BRIEF DESCRIPTION OF THE DRAWINGS

The invention will be further described by way of example with reference to the accompanying drawings, in which:—

FIG. 1(a) to (g) illustrate schematically the prior art concept of dynamic imaging using an imaging agent and the process of analysis;

FIGS. 2(a) to (f) illustrate two sequences of MR images of a contrast-enhanced breast illustrating the techniques of motion correction;

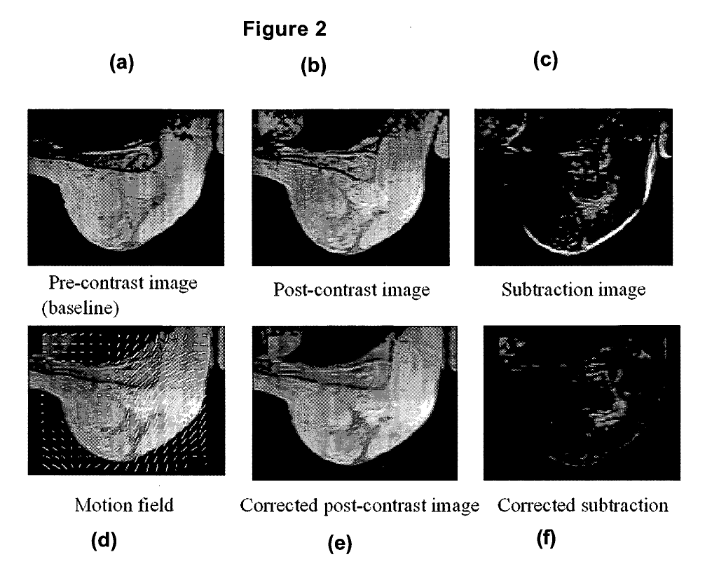

FIG. 3(a) to (f) illustrates in similar fashion to FIG. 1 the verification of the motion correction using the model of imaging agent behaviour;

FIG. 4 illustrates a schematic flow diagram of the process of one embodiment of the invention; and

FIG. 5 illustrates the application of one embodiment of the invention to a recalculation of motion correction.

### DETAILED DESCRIPTION

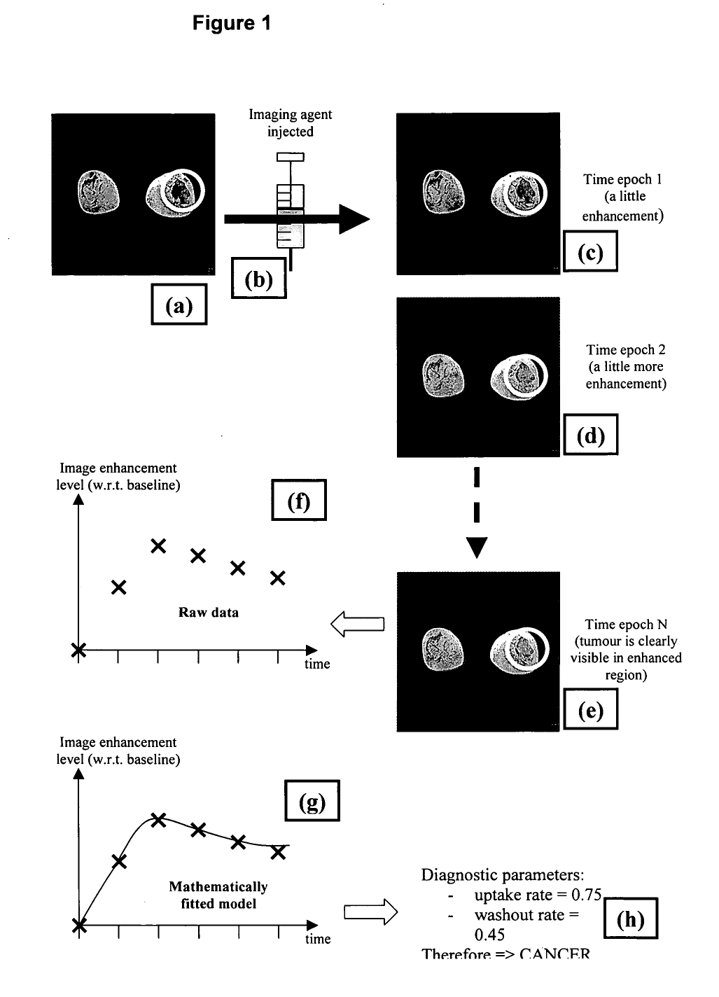

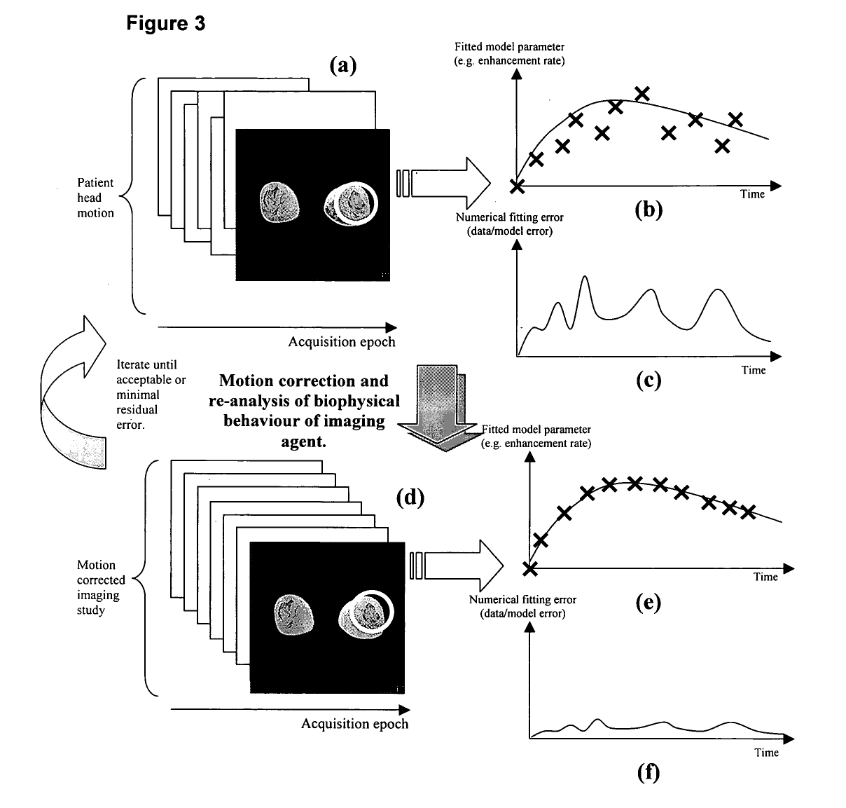

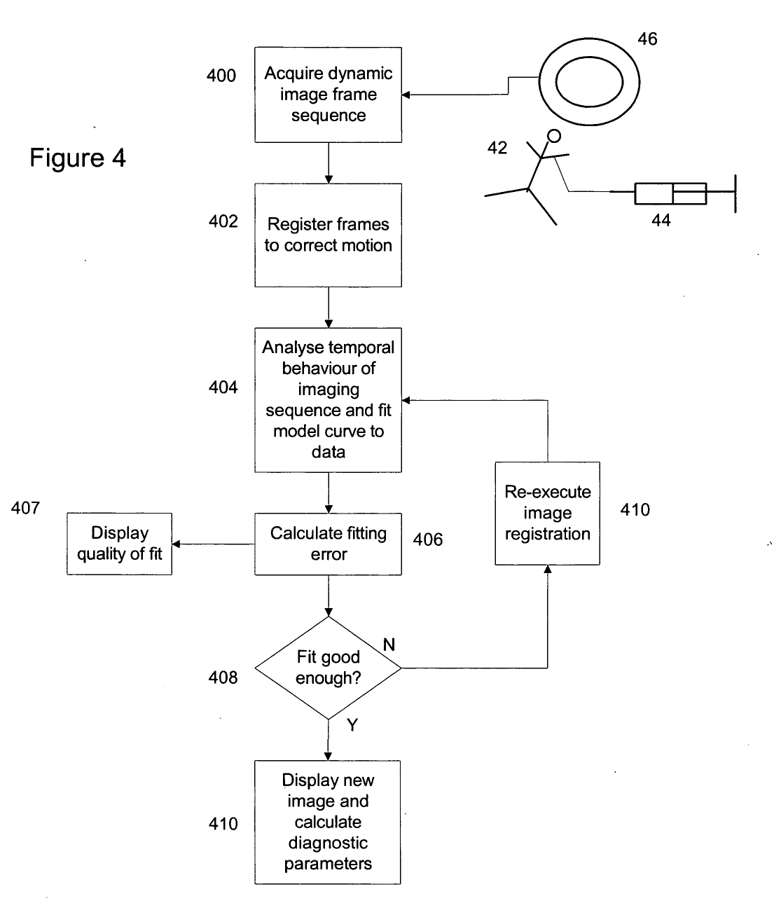

Referring to FIGS. 3 and 4 an embodiment of the invention based on the MR imaging technique of FIG. 1 will be described. Firstly in step 400 of FIG. 4 a dynamic image sequence is acquired from a patient 42 using an imaging apparatus 46 during injection of an imaging agent 44 such as Gadolinium DTPA (Diethyline Triamine Pentaacetic Acid). The sequence is illustrated schematically in FIG. 3(a). It is assumed that there is some patient head motion in the imaging sequence. In step 402 a registration process is carried out to correct the patient motion. The technique may be that described in WO 00/57361. Then the temporal behaviour of the enhancement in a particular part of the image is measured in step 404—the values of enhancement against time are plotted as crosses in FIG. 3(b). The continuous line in FIG. 3(b) illustrates the fitted model, for example in this case the fitted model of the enhancement rate for this imaging agent. Comparing this with FIG. 1(g) it can be seen that the fit is quite poor, this being caused by patient movement. FIG. 3(c) illustrates the numerical fitting error calculated in step 406, i.e. the difference between the value of the data point and the fitted curve.

Incidently, it is possible to apply steps 404 and 406 before applying any image registration algorithm, i.e. before step 402.

The amount of error is displayed in step 407, for example as an overlay whose colour and/or intensity indicate the level of error, or as lines whose length indicate the amount of error. This display gives the user an indication of the quality of the motion correction. If the fit is very poor, this is taken to indicate that there is a substantial degree of patient motion. On the other hand, if the fit is good, this indicates that the patient has not moved much. Obviously there may be different qualities of fit for different parts of the image where some parts of the subject moves but not others.

In this embodiment in regions of the image where the fit is not good enough as indicated by step 408, the motion correction is re-executed in step 410 and the model of the temporal behaviour of the imaging agent is fitted again to the data. The motion correction can be done by any of the following, for example:

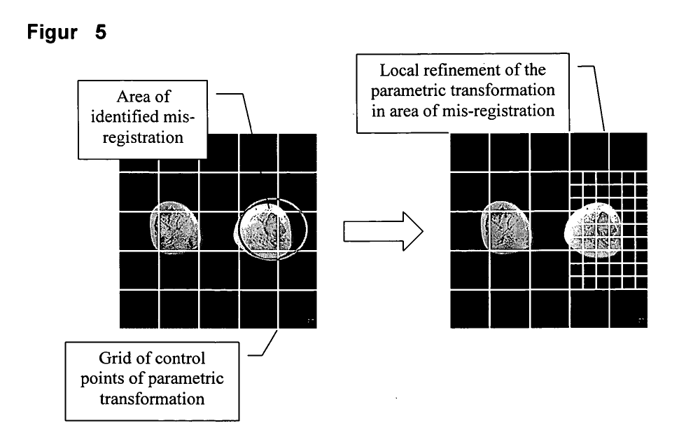

a) addition of extra control points of a parametric transformation in areas or misalignment as shown in FIG. 5;

b) locally loosening stiffness and regularisation constraints in areas of misalignment in the computation of a displacement field;

c) locally changing the scale of the displacement field characteristics; and

d) locally changing the characteristics of local block (size, search, step, etc.) in a block matching technique.

Steps 406, 408 and 410 are repeated iteratively until the residual error is acceptable or at a minimum. This is illustrated in FIGS. 3(d) and (e), where FIG. 3(e) illustrates the corrected data points and the much better fit to the model curve. FIG. 3(f) illustrates the residual error, now much lower than in FIG. 3(c). When the fit is good enough the newly corrected motion image can be displayed in step 412 and the diagnostic parameters such as the wash-in rate and wash-out rate can be calculated taking into account the improved motion correction.

Thus the present invention combines the knowledge about the temporal behaviour of the imaging agent with the motion correction process in order not only to improve the motion correction process and give an indication of its quality, but also to produce ultimately a better motion corrected image which the temporal behaviour model fits, thus allowing a calculation of the diagnostic parameters even in regions of high patient motion.

The imaging agents usable with the present invention are any of those suitable for magnet resonance imaging, computed tomography, positron emission tomography, single photon emission computed tomography, nuclear medicine, ultrasound, x-ray (including angiography) and optical imaging, and include ligands/chelates tagged to paramagnetic compounds (for MRI), radio-labelled tracers/radio pharmaceuticals or nuclear medicine, ionic radiation attenuators for CT and x-ray, receptor-specific imaging agents for cellular behaviour and interactions, genetic markers (radio-labelled, luciferase-labelled etc), blood pool agents and vascular imaging agents.

The invention can be applied to two-dimensional or three-dimensional imaging processes.

What is claimed is:

1. A method of dynamic medical imaging a subject comprising the steps of:—
obtaining a plurality of time separated images of the subject;
registering the plurality of time separated images together to match corresponding locations in the images to each other;
measuring from the registered images the temporal behaviour of an imaged region at a location in the subject; and
comparing the measured temporal behaviour with a model of the expected temporal behaviour of the imaged region to determine the level of agreement therebetween as a measure of the quality of the registration of the time separated images.

2. A method according to claim 1 wherein obtaining the images involves the use of an imaging agent and said model is a model of the temporal behaviour of the imaging agent.

3. A method according to claim 2 wherein the imaging agent is a contrast agent.

4. A method according to claim 1 wherein the level of agreement between the measured temporal behaviour and the expected temporal behaviour is displayed.

5. A method according to claim 4 wherein the level of agreement is displayed overlying an image of the subject.

6. A method according to claim 1 further comprising re-executing the step of registering the plurality of time separated images together in imaged regions where the level of agreement is poor.

7. A method according to claim 6 wherein the registration uses a parameterised process and is re-executed using different registration parameters.

8. A method according to claim 6 wherein the registration is re-executed at a different resolution.

9. A method according to claim 6 wherein the registration is re-executed at a different scale.

10. A method according to claim 6 wherein the registration comprises searching through a search window defined in one of the images and the registration is re-executed using a different sized search window.

11. A method according to claim 1 wherein the model is a temporal model of the take-up and wash-out of an imaging agent administered to the subject.

12. A method according to claim 1 wherein the subject is a living human, animal or plant.

13. A method according to claim 1 wherein the images are acquired by one of magnetic resonance imaging, computed tomography, positron emission tomography, single photon emission computed tomography, nuclear medicine, ultrasound, x-ray and optical imaging.

14. A computer program comprising program code means for executing the steps in claim 1 of registering the plurality of time separated images together to match corresponding locations in the images to each other; measuring from the registered images the temporal behaviour at a location in the subject; and comparing the measured temporal behaviour to a model of the expected temporal behaviour to determine the level of agreement therebetween as a measure of the quality of the registration of the time separated images.

15. A computer readable storage medium carrying a computer program according to claim 14.

16. Dynamic medical image processing apparatus adapted to execute the steps in claim 1 of registering the plurality of time separated images together to match corresponding locations in the images to each other; measuring from the registered images the temporal behaviour of the imaging agent at a location in the subject; and comparing the measured temporal behaviour of the imaging agent to a model of the expected temporal behaviour to determine the level of agreement therebetween as a measure of the quality of the registration of the time separated images.
